(19) United States
(12) Patent Application Publication
(10) Pub. No.: US 2026/0165924 A1
(43) Pub. Date: Jun. 18, 2026

(54) COMPOSITION FOR PROTECTING HAIR SCALP USING ALPHA HYDROXY ACIDS (AHAs)

(71) Applicant: LIW PATENT COMPANY LIMITED BY GUARANTEE, Cork (IE)

(72) Inventor: Tim Meyer-Dulheuer, Cork (IE)

(73) Assignee: LIW PATENT COMPANY LIMITED BY GUARANTEE, Cork (IE)

(21) Appl. No.: 18/986,041

(22) Filed: Dec. 18, 2024

(51) Int. Cl. A61K 8/365; A61K 8/04; A61K 8/34; A61K 8/67; A61Q 5/08

(52) U.S. Cl. CPC A61K 8/365; A61K 8/042; A61K 8/342; A61K 8/676; A61K 8/678; A61Q 5/08

(57) ABSTRACT

The present invention provides a composition for protecting the scalp during oxidative hair coloring or bleaching treatments. The composition comprises alpha-hydroxy acids (AHAs) at concentrations greater than 8%, such as glycolic acid, lactic acid, malic acid, or citric acid, and fatty alcohols like cetyl, stearyl, or cetearyl alcohol. The formulation has a pH range of 8 or greater, ensuring compatibility with oxidative agents without compromising efficacy. Optional stabilizing agents (e.g., carbomers) and antioxidants (e.g., tocopherol) enhance stability and reduce oxidative stress. The gel-form composition, mixed with oxidative agents in a weight ratio of 1:5 to 1:100, minimizes scalp irritation, dryness, and oxidative damage, ensuring even application and long-lasting protection. Suitable for professional and at-home use, it promotes scalp health while preserving hair treatment results.

### FIELD OF THE DISCLOSURE

The present invention relates to compositions for protecting the scalp during oxidative hair coloring or bleaching treatments. Specifically, it pertains to compositions comprising alpha hydroxy acids and fatty alcohols in a pH-optimized formulation, optionally incorporating stabilizing agents and antioxidants, to mitigate scalp irritation, improve comfort, and enhance scalp protection without interfering with the effectiveness of the oxidative agents.

### BACKGROUND

Oxidative hair coloring and bleaching are essential processes in the hair care industry, widely used to alter hair color or lighten natural tones. These treatments rely on powerful chemical agents like hydrogen peroxide and ammonia, which lift hair cuticles, break down melanin pigments, and facilitate the deposition of new pigments. While these chemicals are highly effective in achieving desired results, they are inherently harsh, often causing significant discomfort and damage to the scalp. Scalp irritation, dryness, and oxidative stress are common side effects, making these treatments a challenging experience for many individuals.

One major challenge lies in the high alkalinity and oxidative stress associated with these processes. The oxidative agents used typically operate at a pH range of 8 to 11, weakening the scalp's natural protective barrier. This extreme alkalinity leaves the scalp vulnerable to irritation, inflammation, and even chemical burns. Additionally, oxidative stress caused by free radicals during the treatments can lead to cellular damage and long-term scalp sensitivity, further exacerbating these issues.

Many individuals undergoing these treatments experience scalp irritation, including burning, itching, or redness. For those with pre-existing scalp conditions like dandruff or eczema, the harshness of the chemicals can intensify discomfort and discourage repeated treatments. While traditional protective measures, such as oils, creams, and moisturizers, have been employed to mitigate these issues, they often fall short. These products provide only a superficial barrier and fail to address deeper biochemical issues such as oxidative stress and disrupted PH balance. Furthermore, these traditional solutions can interfere with the chemical processes of hair treatments, potentially diluting active agents and leading to uneven or suboptimal results.

Another limitation is the incompatibility of traditional scalp protectants with hair treatment formulations. Many protectants are not designed to integrate with oxidative processes, and their components can react adversely with the chemical formulations. This can compromise the efficacy and integrity of the hair treatment, creating a need for a solution that provides effective scalp protection while preserving treatment outcomes.

Similarly, bond-building products represent a more recent development in scalp repair technology. These products aim to rebuild the internal disulfide bonds within hair fibers that are broken during chemical processes like bleaching and perming. By restoring these bonds, bond-builders theoretically enhance the structural integrity of scalp from the inside out.

However, bond-building treatments typically involve the use of strong chemicals that can be harsh on hair if not applied under controlled conditions. Many products in this category require exacting pH levels or oxidizing agents, and they are often intended for professional salon use due to the precision required in their application. As a result, they can be inaccessible, expensive, and inconvenient for regular, at-home use.

Several patents and scientific studies have proposed methods for protecting scalp, yet they lack a comprehensive approach to address the scalp irritation and damage without compromising the performance of oxidative hair treatments. For instance, U.S. Pat. No. 11,213,468 describes the method for treating hair using caustic and acidic crosslinking compositions, comprising a Brønsted-base and an organic acid with a cosmetically acceptable carrier. These compositions can be applied to keratin fibers or mixed into hair coloring or bleaching products.

Another patent document KR2,010,010,3964A relates to a hair coloring composition containing Mongolian dandelion extract to minimize scalp irritation and prevent hair damage. It includes ingredients like glycyrrhiza, green tea, and collagen to protect the scalp and minimize skin irritation during hair coloring. It also includes hair-protecting agents like grape or castor oil, ensuring reduced hair damage while providing effective coloring. However, this solution primarily focuses on forming a superficial barrier to prevent scalp irritation but lacks mechanisms to enhance the absorption of active ingredients or improve the scalp's health.

Another patent document DE3534287A1 describes a method to protect the scalp during alkaline hair treatment by applying an aqueous scalp-protecting composition before treatment. This composition forms a barrier layer upon contact with the alkaline treatment, preventing scalp wetting. It contains polymeric carboxylic acids or water-soluble aluminum salts. However, this solution relies on passive protection through a thickened cream or lotion that forms a physical barrier, which may hinder even distribution and compatibility with modern hair treatment formulations but does not address deeper scalp care, such as exfoliation, hydration, or promoting active ingredient absorption.

In another context, while alpha hydroxy acids (AHAs) are widely used in skincare for their exfoliating and hydrating properties, their role in hair care has not been fully explored. AHAs, such as glycolic acid, lactic acid, malic acid, and citric acid, can temporarily open the hair cuticle, allowing active ingredients to penetrate more deeply into the fiber. This makes AHAs suitable as a preparatory step in hair repair treatments, as they can enhance the uptake of subsequent agents that strengthen the internal structure. Further, the efficacy of AHAs in hair care formulations, in high concentration, at pH values of 8 to 11 has not yet been studied and reported. In a pH range from 8 to 11 “AHAs” exist in their deprotonated, anionic form and not as classical “AHAs”.

To date, the use of alpha-hydroxy acids (AHAs) transcends their conventional applications in shampoos and conditioning treatments, where they are primarily utilized to enhance hair's moisture retention and improve shine. This composition innovatively harnesses AHAs for their deeper potential in protecting and repairing the scalp during oxidative hair coloring or bleaching treatments. AHAs gently exfoliate the scalp, removing dead skin cells and preparing it for better absorption of active ingredients, such as fatty alcohols and antioxidants. This exfoliation process facilitates deep penetration of the AHAs, allowing them to hydrate and support cell regeneration effectively, thereby strengthening the scalp's natural protective barrier. Furthermore, by promoting structural repair, AHAs reduce the damage caused by the high alkalinity and oxidative stress of these treatments. The combination of deep absorption and bonding enabled by AHAs enhances the efficacy of other components in the composition, providing long-lasting protection while maintaining the performance of the oxidative agents.

Hence, there is a need for an innovative solution that addresses scalp irritation and damage without compromising the performance of oxidative hair treatments. Such a composition must minimize dryness, irritation, and oxidative stress while integrating seamlessly into the coloring or bleaching process. It should remain stable under typical storage conditions, be easy to mix with treatment products, and enhance the overall health of the scalp by providing hydration, neutralizing oxidative stress, and restoring balance to the scalp's natural defenses, while at the same time not significantly altering the pH value of the hair color and bleach treatment to not negatively impact on their efficacy.

### SUMMARY

An objective of the present disclosure is to protect the scalp from irritation, oxidative damage, and dryness during oxidative hair coloring or bleaching treatments, using a combination of AHAs and fatty alcohols, which gently exfoliate the scalp and promote cell regeneration, with fatty alcohols, which form a protective barrier that locks in moisture and enhances scalp hydration. Together, these components create a balanced formulation that shields the scalp while maintaining the effectiveness of the oxidative treatment process.

Another objective of this invention is to maintain compatibility with oxidative agents, ensuring the hair treatment's performance is not compromised.

Another objective of this invention is to enhance the delivery and retention of active ingredients on the scalp surface.

Another objective of this invention is to optimize the pH to prevent excessive irritation while remaining effective with alkaline oxidative agents.

Another objective of this invention is to provide a stable formulation with optional antioxidants and stabilizing agents to improve efficacy and shelf life.

In an aspect, the present disclosure generally relates to a scalp saver composition and method comprising alpha-hydroxy acids (8%- 20%) for exfoliation and barrier formation, and fatty alcohols for hydration and active ingredient retention. With a pH of 8-11, it ensures compatibility with alkaline agents while minimizing irritation. Stabilizers like carbomers and antioxidants such as tocopherol enhance formulation stability and reduce oxidative stress. Offered as a gel for easy application, it mixes with commercial hair color or bleach in a 1:5 to 1:100 ratio for effective protection and treatment. The ratio is calculated as “grams per gram” of the “scalp saver” formulation added to the commercially available ready-to-use hair color or bleach formulation.

Another aspect of the present invention is to provide a scalp-protecting composition for use during oxidative hair coloring or bleaching treatments, comprising:
i. alpha-hydroxy acids at a concentration greater than 8%; and
ii. fatty alcohols, wherein the pH of said composition ranges from 8-11; and
iii, the addition of the “scalp saver” formulation does not change the pH value of the commercially available hair color or bleach formulation by more than 0.2 units.

In another embodiment, the composition in the present invention further comprises a stabilizing agent selected from, but not limited to carbomers, xanthan gum, or guar gum, or a combination thereof.

In another embodiment, the composition in the present invention is mixed with the oxidative hair color or bleaching agent in a weight ratio of 1:5 to 1:100.

In another embodiment, the composition in the present invention is in the form of a gel.

In another embodiment, the composition in the present invention further comprises an antioxidant selected from, but not limited to tocopherol, ascorbic acid, or plant-based polyphenols or a combination thereof.

In another embodiment, the alpha-hydroxy acid concentration is between 8% and 20%.

In another embodiment, the present invention provides a kit for scalp protection during oxidative hair coloring or bleaching treatments, comprising the composition as described in claim 1; and an instruction manual.

Various objects, features, aspects, and advantages of the inventive subject matter will become more apparent from the following detailed description of preferred embodiments.

### DETAILED DESCRIPTION

The following is a full description of the disclosure's embodiments. The embodiments are described in such a way that the disclosure is clearly communicated. The level of detail provided, on the other hand, is not meant to limit the expected variations of embodiments; rather, it is designed to include all modifications, equivalents, and alternatives that come within the spirit and scope of the current disclosure as defined by the attached claims. Unless the context indicates otherwise, the term “comprise” and variants such as “comprises” and “comprising” throughout the specification are to be read in an open, inclusive meaning, that is, as “including, but not limited to.”

When “one embodiment” or “an embodiment” is used in this specification, it signifies that a particular feature, structure, or characteristic described in conjunction with the embodiment is present in at least one embodiment. As a result, the expressions “in one embodiment” and “in an embodiment” that appear throughout this specification do not necessarily refer to the same embodiment.

Furthermore, in one or more embodiments, the specific features, structures, or qualities may be combined in any way that is appropriate.

Unless the content clearly demands otherwise, the singular terms “a,” “an,” and “the” include plural referents in this specification and the appended claims. Unless the content explicitly mandates differently, the term “or” is normally used in its broad definition, which includes “and/or.”

All processes described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

Various terms as used herein are shown below. To the extent a term used in a claim is not defined below, it should be given the broadest definition persons in the pertinent art have given that term as reflected in printed publications and issued patents at the time of filing.

### Definitions

For the purpose of the present disclosure, Alpha-Hydroxy Acids (AHAs) may be defined as Organic acids with one or more hydroxyl groups attached to the alpha carbon. Commonly used in skincare for exfoliation, AHAs, such as glycolic acid, lactic acid, malic acid, and citric acid, are used in this invention for their ability to penetrate the hair cuticle and facilitate deeper absorption of subsequent treatments. For the purpose of the present disclosure, Alpha-Hydroxy-Acids are also referred to as AHAs also in the alkaline pH range, for example at a pH range of 8 to 11, although they will then be deprotonated and present in their anionic form.

Alpha hydroxy acids (AHAs), such as glycolic acid, lactic acid, malic acid, and citric acid, individually or in combination, at an optimized concentration of 8% to 20% to balance efficacy and safety. AHAs provide gentle exfoliation, removing dead skin cells from the scalp's surface to enhance absorption of active ingredients like fatty alcohols and antioxidants, promote cell turnover, and improve scalp health. Glycolic acid, with its small molecular size, penetrates deeply for effective exfoliation and hydration; lactic acid offers milder exfoliation suitable for sensitive scalps while boosting moisture retention; and citric acid balances the formulation's pH and provides antioxidant benefits, enhancing overall scalp regeneration.

For the purpose of the present invention, fatty alcohols such as cetyl alcohol, stearyl alcohol, or cetearyl alcohol, play a crucial role in maintaining scalp health and enhancing the efficacy of active ingredients. These fatty alcohols form a protective barrier on the scalp, preventing irritants from penetrating while locking in moisture to keep the scalp hydrated and reduce irritation, particularly from the alkaline conditions of oxidative treatments. Additionally, they facilitate the controlled release of AHAs and antioxidants, ensuring a gradual and sustained action of these active components. By balancing hydration and protection, fatty alcohols enhance the absorption of AHAs and antioxidants, allowing deeper penetration of beneficial ingredients without compromising the scalp's natural barrier.

For the purpose of the present invention, stabilizing agents like carbomers, xanthan gum, hydroxyethylcellulose-polymers, and guar gum are crucial components in hair care formulations. They not only enhance the product's texture but also ensure its stability and usability. Carbomers, for instance, provide excellent thickening properties, giving the composition a gel-like consistency that adheres well to the scalp. Hydroxyethylcellulose-polymers are known for their excellent gel-forming characteristics over a wide range of pH values, typically pH 2 to 12. Natural stabilizers such as xanthan gum and guar gum further improve the product's viscosity and spread-ability, making it easy to mix with other hair care products and ensuring smooth application. By preventing ingredient separation during storage and application, these agents maintain a uniform distribution of active ingredients like AHAs, fatty alcohols, and antioxidants, maximizing their efficacy. Overall, stabilizing agents play a vital role in optimizing the performance and user experience of hair care products.

For the purpose of the present invention, antioxidants, such as tocopherol (Vitamin E), ascorbic acid (Vitamin C), and plant-based polyphenols, are essential components in hair care formulations. During oxidative hair treatments like coloring or bleaching, free radicals are generated, causing oxidative stress and damaging the scalp. These antioxidants effectively neutralize these harmful free radicals, minimizing cellular damage and promoting scalp health. Tocopherol, a lipid-soluble antioxidant, protects scalp cells from oxidative damage and helps restore the skin's barrier. Ascorbic acid, a water-soluble antioxidant, enhances collagen synthesis and supports the scalp's natural repair mechanisms. Plant-based polyphenols, derived from natural sources, provide additional antioxidant protection and nourishment to the scalp. By incorporating these powerful antioxidants, hair care formulations can help maintain a healthy scalp and minimize the adverse effects of oxidative hair treatments.

For the purpose of the present disclosure, the pH of the composition is carefully optimized to a range of 8 to 11. This pH level ensures compatibility with the high alkalinity of hair coloring and bleaching agents, allowing for a seamless integration and preventing adverse chemical reactions that could hinder the oxidative process.

For the purpose of the present invention, the composition, presented in a convenient gel form, offers enhanced functionality and ease of use. This gel consistency ensures effortless mixing with oxidative hair coloring or bleaching agents, enabling a uniform and controlled application. The gel's ability to adhere well to the scalp prevents runoff, maximizing the protective effect. This innovative formulation not only simplifies the hair care process but also optimizes the delivery of essential ingredients, promoting scalp health and minimizing the adverse effects of oxidative hair treatments.

For the purpose of the present invention, the composition is designed to be mixed with oxidative hair coloring or bleaching agents in a weight ratio ranging from 1:5 to 1:100. This careful balance ensures adequate scalp protection without compromising the efficacy of the oxidative agents. By maintaining optimal hair treatment performance, this formulation provides a comprehensive solution for protecting the scalp during hair coloring and bleaching processes.

For the purpose of the present invention, Fatty Alcohol Ester may be defined as type of compound often derived from the esterification of fatty acids and alcohols. These esters enhance the formulation's spread-ability, moisturizing properties, and overall smooth feel on hair, aiding in the product's performance and user experience.

In an embodiment, the present disclosure provides a composition for protecting hair scalp, comprising:
i. alpha-hydroxy acids at a concentration greater than 8%; and
ii. fatty alcohols, wherein pH of said composition ranges from 8-11; and
iii, the addition of the “sculp saver” formulation does not change the pH value of the commercially available hair color or bleach formulation by more than 0.2 units.

In another embodiment, the alpha-hydroxy acids are selected from, but not limited to glycolic acid, lactic acid, malic acid, or citric acid or a combination thereof.

In another embodiment, the fatty alcohols are selected from, but not limited to cetyl alcohol, stearyl alcohol, or cetearyl alcohol, or a combination thereof, enhancing the delivery and retention of active ingredients on the scalp surface.

In another embodiment, a stabilizing agent is selected from, but not limited to carbomers, hydroxyethylcellulose-polymer, xanthan gum, or guar gum, or a combination thereof.

In another embodiment, the composition is mixed with the oxidative hair color or bleaching agent in a weight ratio of 1:5 to 1:100.

In another embodiment, the composition is in the form of a gel.

In another embodiment, the composition further comprises an antioxidant selected from, but not limited to tocopherol, ascorbic acid, or plant-based polyphenols, or a combination thereof.

In another embodiment, the present invention comprises the alpha-hydroxy acid concentration is between 8% and 20%.

In another embodiment, the pH of the composition is in the range of 8 to 11.

In another embodiment, the present invention provides a kit for scalp protection during oxidative hair coloring or bleaching treatments, comprising the composition as described in claim 1; and an instruction manual.

In an exemplary embodiment, the present disclosure may provide a method of applying an aqueous alpha-hydroxy acid-based composition with a concentration exceeding 8%, potentially in combination with fatty alcohols, at a pH of 8 or greater.

Accordingly, this invention is designed to address the limitations of existing scalp protection products by providing a composition and method that not only shields the scalp during oxidative hair treatments but also enhances scalp health by reducing irritation and oxidative damage. By utilizing alpha-hydroxy acids (AHAs) to gently exfoliate and prepare the scalp, combined with fatty alcohols to form a protective barrier, the composition ensures deeper penetration of active ingredients and promotes effective hydration. This innovative approach provides long-lasting protection and improved scalp resilience without compromising the efficacy of hair coloring or bleaching treatments.

It is also notable that the disclosed composition herein avoids harsh chemicals like strong oxidizing or alkalizing agents, which often cause scalp irritation and sensitivity. Instead, it uses a balanced mix of AHAs and fatty alcohols to protect and hydrate the scalp during oxidative hair treatments. This gentle, safe formulation reduces irritation and damage, making it suitable for regular at-home use while meeting both professional and consumer needs.

Moreover, the composition and method disclosed for scalp protection deliver long-lasting results, ensuring that the scalp remains healthy, hydrated, and irritation-free during and after oxidative hair treatments. This protective formulation not only minimizes immediate discomfort but also enhances the scalp's resilience against environmental stressors and chemical damage. Unlike conventional scalp protectors, the effects of this invention, such as reduced sensitivity and improved scalp condition, persist across multiple hair treatments, offering durable and reliable protection.

Hence, composition for protecting a scalp during oxidative hair coloring or bleaching treatments and method presented offers a solution by combining alpha-hydroxy acids (AHAs), fatty alcohols, and optional antioxidants and stabilizers. This unique composition mitigates alkaline damage, as AHAs in optimized concentrations gently exfoliate the scalp, promote cell regeneration, and counteract the adverse effects of high pH. Fatty alcohols form a protective barrier on the scalp, locking in moisture and preventing irritants from penetrating the skin. Antioxidants, such as tocopherol or ascorbic acid, neutralize free radicals generated during the treatment process, reducing oxidative stress and minimizing cellular damage. Importantly, the composition is chemically compatible with oxidative agents, preserving the activity of coloring or bleaching agents to ensure even and effective results. The gel form of the composition further enhances its application, allowing it to mix easily with oxidative products and ensuring uniform coverage.

While the foregoing describes various embodiments of the disclosure, other and further embodiments of the disclosure may be devised without departing from the basic scope thereof. The scope of the invention is determined by the claims that follow. The invention is not limited to the described embodiments, versions, or examples, which are included to enable a person having ordinary skill in the art to make and use the invention when combined with information and knowledge available to the person having ordinary skill in the art.

### EXAMPLES

The present invention is further explained in the form of the following examples. However, it is to be understood that the following examples are merely illustrative and are not to be taken as limitations upon the scope of the invention.

### Materials

Alpha-Hydroxy Acids (AHAs): Alpha-hydroxy acids such as glycolic acid, lactic acid, malic acid, and citric acid are incorporated into the composition in concentrations ranging from 8% to 20%. These acids are known to gently exfoliate the scalp, helping to loosen dead skin cells and facilitating deeper penetration of active ingredients. The AHAs also help in balancing the scalp's pH, enhancing comfort during oxidative hair coloring or bleaching treatments.

Fatty Alcohols: Fatty alcohols like cetyl alcohol, stearyl alcohol, or cetearyl alcohol are used to create a barrier on the scalp, locking in moisture and reducing irritation. These fatty alcohols also support the even distribution of the composition across the scalp, ensuring maximum coverage and enhancing the retention of AHAs and other active ingredients.

Stabilizing Agents: Carbomers, hydroxyethylcellulose-polymers, xanthan gum, or guar gum are added to the formulation to improve the texture and stability of the composition. These agents ensure that the gel remains consistent, allowing for smooth application and preventing the separation of ingredients over time.

Antioxidants: Tocopherol (Vitamin E), ascorbic acid (Vitamin C), or plant-based polyphenols are included in the composition to neutralize free radicals produced during oxidative hair treatments. These antioxidants help to minimize oxidative stress on the scalp, maintaining its health and comfort throughout the coloring or bleaching process.

Sample Preparation: Four gel-samples formulations (1-4) of different AHAs, Fatty Alcohols, and Stabilizing Agents, in water, were created and their pH values adjusted using a 0,1-molar sodium hydroxide (NaOH) solution.

| | Formu- | Formu- | Formu- | Formu- |
|---|---|---|---|---|
| | lation 1 | lation 2 | lation 3 | lation 4 |
| Solvent | Water | Water | Water | Water |
| AHA | Glycolic | Malic | Lactic | Citric |
| | Acid 8.3% | Acid 12.6% | Acid 13.1% | Acid 18.4% |
| Fatty | Cetyl | Cetearyl | Stearyl | Cetearyl |
| Alcohol | Alcohol | Alcohol | Alcohol | Alcohol |
| Stabilizing | Carbomer | HydroxyEthyl | Xanthan Gum | Guar Gum |
| Agent | | Cellulose | | |
| pH Modifier | NaOH | NaOH | NaOH | NaOH |
| pH Value | 9 | 9 | 10 | 11 |

Test Execution 1: The hair of sixteen human test candidates (C1-C16) were washed with a deep cleansing shampoo (Goldwell Scalp Specialist Deep Cleansing Shampoo) and afterwards blow-dried. Such human test candidates were then treated with the compositions in a “half-head” execution where on the right side of the respective candidates Formulations 1-4, mixed with commercially available hair color or bleach, were applied and on the respective left side the control formulation was applied, comprising of the respective native hair color or bleach formulation without Formulations 1-4. All hair color and bleach mixes, with and without Formulations 1-4, let side as well as right side, were left on as per the instructions of the respective hair color and hair bleach manufactures. Subsequently, the full head was rinsed, shampooed with the same deep cleansing shampoo and the hair blow-dried.

Test Execution 2: Identical to test execution 1, the hair of other sixteen human test candidates (C17-C32) were washed with a deep cleansing shampoo (Goldwell Scalp Specialist Deep Cleansing Shampoo) and afterwards blow-dried. Test candidates C17-C32 were also treated with the compositions in a “half-head” execution where on the right side of the respective candidates Formulations 1˜4 were applied and left on 10 minutes, before commercially available hair color or bleach formulations were applied to the right and left side of the respective head. The left side is the control. All hair color and bleach mixes were left on as per the instructions of the respective hair color and hair bleach manufactures. Subsequently, the full head was rinsed, shampooed with the same deep cleansing shampoo and the hair blow-dried.

Results Test Execution 1: After the test execution, Candidate C1-C16 were asked to evaluate the right and left side of their respective treatment with regards to their hair color (if applicable), their hair bleach (if applicable), and their scalp health, determined as absence of scalp irritation and burning. The following table summarizes the results:

| | | Color (C) | Preference Right | Preference Right |
|---|---|---|---|---|
| | | or | (R), Left (L), No | (R), Left (L), No |
| Candidate | Formula | Bleach(B) | Preference (NP) | Preference (NP) |
| C1 | 1 | C | R | R |
| C2 | 2 | B | NP | R |
| C3 | 3 | C | NP | R |
| C4 | 4 | B | R | R |
| C5 | 1 | B | NP | R |
| C6 | 2 | C | NP | R |
| C7 | 3 | B | NP | R |
| C8 | 4 | C | NP | R |
| C9 | 1 | C | NP | R |
| C10 | 2 | B | R | R |
| C11 | 3 | C | R | R |
| C12 | 4 | B | NP | R |
| C13 | 1 | B | NP | R |
| C14 | 2 | C | NP | R |
| C15 | 3 | B | NP | R |
| C16 | 4 | C | NP | R |

Results Test Execution 2: After the test execution, Candidate C16-C32 were asked to evaluate the right and left side of their respective treatment with regards to their hair color (if applicable), their hair bleach (if applicable), and their scalp health, determined as absence of scalp irritation and burning. The following table summarizes the results:

| | | Color (C) | Preference Right | Preference Right |
|---|---|---|---|---|
| | | or | (R), Left (L), No | (R), Left (L), No |
| Candidate | Formula | Bleach(B) | Preference (NP) | Preference (NP) |
| C17 | 1 | C | NP | R |
| C18 | 2 | B | NP | R |
| C19 | 3 | C | R | R |
| C20 | 4 | B | NP | R |
| C21 | 1 | B | R | R |
| C22 | 2 | C | NP | R |
| C23 | 3 | B | NP | R |
| C24 | 4 | C | NP | R |
| C25 | 1 | C | NP | R |
| C26 | 2 | B | NP | R |
| C27 | 3 | C | NP | R |
| C28 | 4 | B | NP | R |
| C29 | 1 | B | NP | R |
| C30 | 2 | C | R | R |
| C31 | 3 | B | NP | R |
| C32 | 4 | C | R | R |

Durability: The protective effects of the composition were retained after five wash cycles, with no significant irritation or degradation in the protection, showing that the formulation offers long-lasting benefits.

Safety: Dermatological testing confirmed that the composition is safe for use on the scalp, with no signs of irritation, redness, or discomfort, ensuring its suitability for both professional and at-home use.

These examples illustrate various embodiments of the invention, demonstrating its flexibility and effectiveness in treating and preventing

Dermatological and joint conditions. Different formulations and administration methods cater to different user preferences and therapeutic goals.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Advantages of the Present Invention

The present invention provides the composition that offers an enhanced scalp protection by shielding the scalp from irritation and oxidative stress during hair coloring or bleaching. It ensures the efficacy of hair treatment agents while prioritizing scalp comfort and safety. The gel form and specified mixing ratio make the composition user-friendly, suitable for both professional and at-home use. The inclusion of stabilizers and antioxidants guarantees long-term product stability. This versatile formulation caters to diverse hair treatment needs, making it a comprehensive solution for scalp protection:

### Enhanced Scalp Barrier Function:

The combination of fatty alcohols such as cetyl alcohol, stearyl alcohol, or cetearyl alcohol with alpha-hydroxy acids (AHAs) provides a dual-action technical advantage for the scalp. Fatty alcohols create a semi-occlusive barrier that locks in moisture and prevents the penetration of irritants, while AHAs promote gentle exfoliation and cell turnover. This synergy strengthens the scalp's natural barrier function, reducing sensitivity and improving its resilience against the high alkalinity and oxidative stress caused by hair treatments.

### Effective Scalp Protection:

The composition significantly reduces scalp irritation, oxidative stress, and dryness caused by hair coloring or bleaching treatments. AHAs gently exfoliate and hydrate the scalp, while fatty alcohols form a protective barrier, minimizing direct contact with harsh chemicals and enhancing comfort during the treatment process.

Compatibility with Oxidative Agents:

The formulation is designed to integrate seamlessly with oxidative hair coloring and bleaching agents. Its optimized pH and carefully selected ingredients maintain the efficacy of the chemical processes, ensuring even coloring and bleaching results without compromising the treatment's performance.

### Enhanced Penetration and Retention of Active Ingredients:

The synergy of AHAs and fatty alcohols promotes deep penetration and sustained retention of active ingredients on the scalp. This ensures that antioxidants, moisturizers, and other beneficial components work effectively to repair and protect the scalp during and after the hair treatment.

### Improved Stability and Longevity:

The inclusion of stabilizing agents like carbomers, xanthan gum, or guar gum ensures that the composition remains homogeneous and effective under ambient storage conditions for up to 12 months. This stability enhances the product's reliability for both professional and consumer use.

### User-Friendly Application:

The gel form of the composition, combined with an optimized mixing ratio (1:5 to 1:100), simplifies its application with oxidative treatments. Its ease of use makes it suitable for both salon professionals and at-home consumers, ensuring a convenient and comfortable user experience.

We claim:

1. A composition for protecting a scalp during oxidative hair coloring or bleaching treatments, comprising:
i alpha-hydroxy acids in a concentration greater than 8%; and
ii fatty alcohols, wherein pH of said composition is greater than or equal to 8.

2. The composition of claim 1, wherein the alpha-hydroxy acids are selected from, but not limited to glycolic acid, lactic acid, malic acid or citric acid or a combination thereof.

3. The composition of claim 1, wherein the fatty alcohols are selected from, but not limited to cetyl alcohol, stearyl alcohol, or cetearyl alcohol, or a combination thereof, enhancing the delivery and retention of active ingredients on the scalp surface.

4. The composition of claim 1, further comprising a stabilizing agent selected from, but not limited to carbomers, hydroxyethylcellulose-polymers, xanthan gum, or guar gum, or a combination thereof.

5. The composition of claim 1, wherein the composition is mixed with an oxidative hair color or bleaching agent in a weight ratio of 1:5 to 1:100.

6. The composition of claim 1, wherein the composition is in the form of a gel.

7. The composition of claim 1, wherein the composition further comprises an antioxidant selected from, but not limited to tocopherol, ascorbic acid, or plant-based polyphenols, or a combination thereof.

8. The composition of claim 1, wherein the alpha-hydroxy acid concentration is between 8% and 20%.

9. The composition of claim 1, wherein the pH of the composition is in the range of 8 to 11.

10. A method for protecting the scalp during oxidative hair coloring or bleaching treatments, wherein the composition as defined in claim 1 is combined with an oxidative hair color or bleaching formulation to form a mixture, wherein the mixture is subsequently applied to the scalp and hair to minimize irritation and damage.

11. A method for protecting the scalp, wherein the composition as defined in claim 1 is applied prior the application of an oxidative hair color or bleaching formulation.

12. A method for protecting the scalp, wherein the composition as defined in claim 1 is applied and left on the scalp at least 10 minutes prior the application of an oxidative hair color or bleaching formulation.

13. A kit for scalp protection during oxidative hair coloring or bleaching treatments, comprising:
the composition as described in claim 1; and
an instruction manual.
